(19) United States
(12) Patent Application Publication
(10) Pub. No.: US 2012/0165639 A1
(43) Pub. Date: Jun. 28, 2012

(54) STORAGE OF CALIBRATION DATA AT A CONTINUOUS GLUCOSE MONITOR

(75) Inventors: Timothy Peter Engelhardt, Avon, IN (US); Guenter Handwerker, Boehl-Iggelheim (DE); Mark Nierzwick, Brownsburg, IN (US); Gerhard Frisch, Edingen-Neckarhausen (DE); Phillip E. Pash, Indianapolis, IN (US)

(73) Assignee: ROCHE DIAGNOSTICS OPERATIONS, INC., Indianapolis, IN (US)

(21) Appl. No.: 12/975,792

(22) Filed: Dec. 22, 2010

(51) Int. Cl. A61B 5/145

(52) U.S. Cl. 600/365

(57) ABSTRACT

A method for storing data at a continuous glucose monitor is presented. The method includes measuring the blood glucose level of the patient. The method also includes receiving a plurality of physical attributes related to the glucose level of the patient. The method further includes determining calibration data based on the measured blood glucose level and at least one of the plurality of physical attribute samples, the calibration data configured to allow the handheld diabetes managing device to determine the estimated glucose level of the patient based on the plurality of physical attribute samples. Finally, the method includes transmitting the calibration data from the handheld diabetes managing device to the continuous glucose monitor for storage at the continuous glucose monitor. The calibration data and plurality of physical attribute samples can be retrieved by a separate device to determine an estimated glucose level of the patient.

### FIELD

The present disclosure relates generally to medical devices and more particularly to a system and method for ensuring that data stored at a continuous glucose monitor can be utilized to estimate a patient's glucose level.

### BACKGROUND

Medical devices are often used as diagnostic devices and/or therapeutic devices in diagnosing and/or treating medical conditions of patients. For example, a blood glucose meter is used as a diagnostic device to measure blood glucose levels of patients suffering from diabetes. An insulin infusion pump is used as a therapeutic device to administer insulin to patients suffering from diabetes.

Diabetes mellitus, often referred to as diabetes, is a chronic condition in which a person has elevated blood glucose levels that result from defects in the body's ability to produce and/or use insulin. There are three main types of diabetes. Type 1 diabetes can be autoimmune, genetic, and/or environmental and usually strikes children and young adults. Type 2 diabetes accounts for 90-95% of diabetes cases and is linked to obesity and physical inactivity. Gestational diabetes is a form of glucose intolerance diagnosed during pregnancy and usually resolves spontaneously after delivery.

In 2009, according to the World Health Organization, at least 220 million people worldwide suffer from diabetes. In 2005, an estimated 1.1 million people died from diabetes. The incidence of diabetes is increasing rapidly, and it is estimated that between 2005 and 2030, the number of deaths from diabetes will double. In the United States, nearly 24 million Americans have diabetes, and an estimated 25% of seniors age 60 and older are affected. The Centers for Disease Control and Prevention forecast that 1 in 3 Americans born after 2000 will develop diabetes during their lifetime. The National Diabetes Information Clearinghouse estimates that diabetes costs $132 billion in the United States alone every year. Without treatment, diabetes can lead to severe complications such as heart disease, stroke, blindness, kidney failure, amputations, and death related to pneumonia and flu.

Diabetes is managed primarily by controlling the level of glucose in the bloodstream. This level is dynamic and complex, and is affected by multiple factors including the amount and type of food consumed, and the amount of insulin (which mediates transport of glucose across cell membranes) in the blood. Glucose levels are also sensitive to exercise, sleep, stress, smoking, travel, illness, menses, and other psychological and lifestyle factors unique to individual patients. The dynamic nature of blood glucose and insulin, and all other factors affecting blood glucose, often require a person with diabetes to forecast blood glucose levels. Therefore, therapy in the form of insulin or oral medications, or both, can be timed to maintain blood glucose levels in an appropriate range.

Management of diabetes is time-consuming for patients because of the need to consistently obtain reliable diagnostic information, follow prescribed therapy, and manage lifestyle on a daily basis. Diagnostic information, such as blood glucose, is typically obtained from a capillary blood sample with a lancing device and is then measured with a handheld blood glucose meter. Interstitial glucose levels may be obtained from a continuous glucose sensor worn on the body. Prescribed therapies may include insulin, oral medications, or both. Insulin can be delivered with a syringe, an ambulatory infusion pump, or a combination of both. With insulin therapy, determining the amount of insulin to be injected can require forecasting meal composition of fat, carbohydrates and proteins along with effects of exercise or other physiologic states. The management of lifestyle factors such as body weight, diet, and exercise can significantly influence the type and effectiveness of a therapy.

Management of diabetes involves large amounts of diagnostic data and prescriptive data acquired in a variety of ways: from medical devices, from personal healthcare devices, from patient-recorded logs, from laboratory tests, and from healthcare professional recommendations. Medical devices include patient-owned bG meters, continuous glucose monitors, ambulatory insulin infusion pumps, diabetes analysis software, and diabetes device configuration software. Each of these systems generates and/or manages large amounts of diagnostic and prescriptive data. Personal healthcare devices include weight scales, blood pressure cuffs, exercise machines, thermometers, and weight management software. Patient recorded logs include information relating to meals, exercise and lifestyle. Lab test results include HbA1C, cholesterol, triglycerides, and glucose tolerance. Healthcare professional recommendations include prescriptions, diets, test plans, and other information relating to the patient's treatment.

There is a need for a handheld device to aggregate, manipulate, manage, present, and communicate diagnostic data and prescriptive data from medical devices, personal healthcare devices, patient recorded information, biomarker information, and recorded information in an efficient manner. The handheld device can improve the care and health of a person with diabetes so that the person with diabetes can lead a full life and reduce the risk of complications from diabetes.

Additionally, to effectively manage the care and health of the patient, there is a need for the handheld device to communicate with and process information received from other medical devices and systems. A handheld device may receive patient information from a number of different sources, such as an insulin pump, a continuous glucose monitor, a computer program, user input, etc. In order to accurately utilize this information, the handheld device may need to calibrate the information received from these sources. For example, a handheld diabetes managing device may receive, from a continuous glucose monitor, raw data that is related to a blood glucose level of a patient. In order to make use of this raw data, the handheld diabetes managing device may need to be calibrated to correlate the received raw data with a measured blood glucose level of the patient. The accuracy of this calibration can affect the care and treatment of the patient. In the event that the handheld diabetes managing device malfunctions or is otherwise unavailable, the raw data generated by and stored in the continuous glucose monitor may become unusable. Accordingly, there is a need for a system and method of ensuring the usability of raw data generated by a continuous glucose monitor to determine an accurate estimated glucose level of a patient.

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

According to the present disclosure, a method for storing data at a continuous glucose monitor and a handheld diabetes managing device such that an estimated glucose level of a patient can be determined by a third device from the data stored at the continuous glucose monitor is presented. The method includes measuring the blood glucose level of the patient. The method also includes receiving a plurality of physical attributes related to the glucose level of the patient. The method further includes determining calibration data based on the measured blood glucose level and at least one of the plurality of physical attribute samples, the calibration data configured to allow the handheld diabetes managing device to determine the estimated glucose level of the patient based on the plurality of physical attribute samples. Finally, the method includes transmitting the calibration data from the handheld diabetes managing device to the continuous glucose monitor for storage at the continuous glucose monitor.

According to the present disclosure, a method for storing data at a continuous glucose monitor and a handheld diabetes managing device such that an estimated glucose level of a patient can be determined by a third device from the data stored at the continuous glucose monitor is presented. The method includes sampling a physical attribute related to a glucose level of the patient with the continuous glucose monitor to generate a plurality of physical attribute samples. The method further includes storing the plurality of physical attribute samples at the continuous glucose monitor and associating each of the plurality of physical attribute samples with a time indicator. The method also includes measuring the blood glucose level of the patient with the handheld diabetes managing device and transmitting the plurality of physical attribute samples to the handheld diabetes managing device. Additionally, the method includes determining calibration data at the handheld diabetes managing device based on the measured blood glucose level and at least one of the plurality of physical attribute samples. The calibration data is configured to allow the handheld diabetes managing device to determine the estimated glucose level of the patient based on the plurality of physical attribute samples. The method can also include transmitting the calibration data from the handheld diabetes managing device to the continuous glucose monitor, storing the calibration data at the continuous glucose monitor, transmitting the calibration data to the third device, transmitting the plurality of physical attribute samples to the third device and determining the estimated glucose level of the patient at the third device based on the plurality of physical attribute samples and the calibration data.

A diabetes management system that allows a separate device to determine an estimated glucose level of a patient is also presented. The diabetes management system can include a continuous glucose monitor and a handheld diabetes managing device. The continuous glucose monitor can include a memory and be configured to: (i) sample a physical attribute related to a glucose level of the patient to generate a plurality of physical attribute samples, (ii) store the plurality of physical attribute samples and (iii) store calibration data configured to allow the separate device to determine the estimated glucose level of the patient based on the plurality of physical attribute samples. The handheld diabetes managing device can be in communication with the continuous glucose monitor and be configured to: (i) receive the plurality of physical attribute samples from the continuous glucose monitor, (ii) measure the blood glucose level of the patient, (iii) determine the calibration data based on the measured blood glucose level and at least one of the plurality of physical attribute samples, and (iv) transmit the calibration data to the continuous glucose monitor for storage.

Further areas of applicability of the present disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows a patient and a treating clinician;

FIG. 2 shows a patient with a continuous glucose monitor (CGM), ambulatory durable insulin infusion pump, ambulatory non-durable insulin infusion pump, and diabetes manger;

FIG. 3 shows a diabetes care system of systems used by patients and clinicians to manage diabetes;

FIG. 4 is a functional block diagram of a diabetes manager;

FIG. 5 is a functional block diagram of a continuous glucose monitor;

FIG. 6 shows a flow-chart illustrating an exemplary method of storing data at a continuous glucose monitor according to the present disclosure; and

FIG. 7 is a functional block diagram of an exemplary memory of the continuous glucose monitor of FIG. 5.

### DETAILED DESCRIPTION

Referring now to FIG. 1, a person 100 with diabetes and a healthcare professional 102 are shown in a clinical environment. Persons with diabetes include persons with metabolic syndrome, pre-diabetes, type 1 diabetics, type 2 diabetics, and gestational diabetics and are collectively referred to as a patient. Healthcare providers for diabetes are diverse and include nurses, nurse practitioners, physicians, and endocrinologists and are collectively referred to as a clinician.

During a healthcare consultation, the patient 100 typically shares with the clinician 102 a variety of patient data including blood glucose measurements, continuous glucose monitor data, amounts of insulin infused, amounts of food and beverages consumed, exercise schedules, and other lifestyle information. The clinician 102 can obtain additional patient data that includes measurements of HbA1C, cholesterol levels, triglycerides, blood pressure, and weight of the patient 100. The patient data can be recorded manually or electronically on a handheld diabetes managing device 104, a diabetes analysis software executed on a personal computer (PC) 106, and/or a web-based diabetes analysis site (not shown). The clinician 102 can analyze the patient data manually or electronically using the diabetes analysis software and/or the web-based diabetes analysis site. After analyzing the patient data and reviewing adherence of the patient 100 to previously prescribed therapy, the clinician 102 can decide whether to modify the therapy for the patient 100.

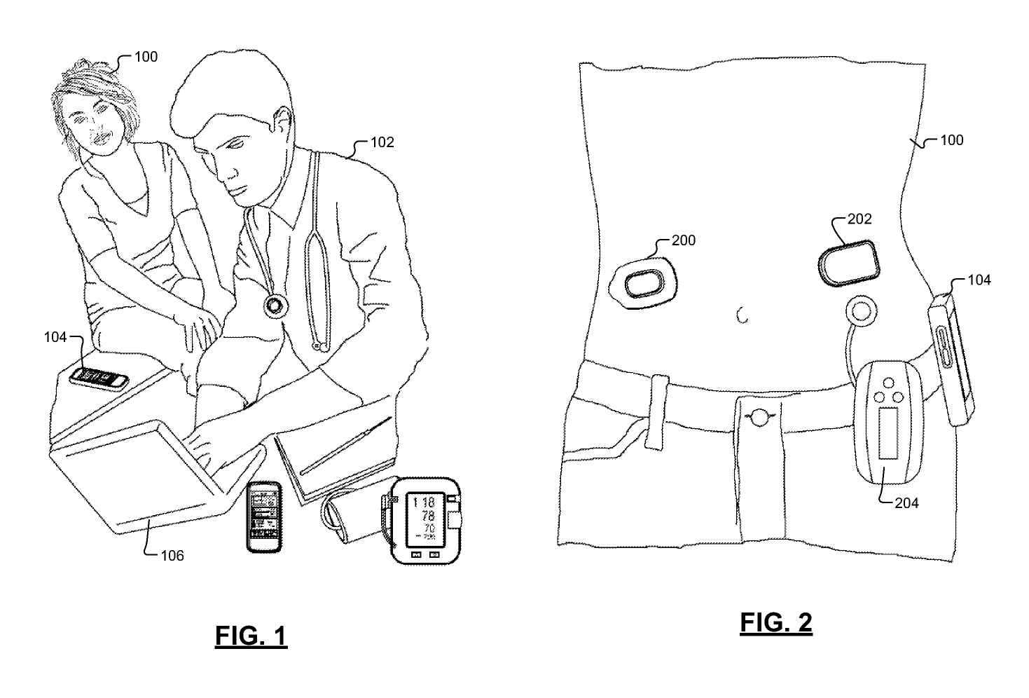

Referring now to FIG. 2, the patient 100 can use a continuous glucose monitor (CGM) 200, an ambulatory durable insulin infusion pump 202 or an ambulatory non-durable insulin infusion pump 204 (collectively insulin pump 202 or 204), and the handheld diabetes managing device 104 (hereinafter the diabetes manager 104). The CGM 200 uses a subcutaneous sensor to sense and monitor the amount of glucose in the blood of the patient 100 and communicates corresponding readings to the handheld diabetes managing device 104.

The diabetes manager 104 performs various tasks including measuring and recording blood glucose levels, determining an amount of insulin to be administered to the patient 100 via the insulin pump 202 or 204, receiving patient data via a user interface, archiving the patient data, etc. The diabetes manager 104 periodically receives readings from the CGM 200 indicating glucose level in the blood of the patient 100. The diabetes manager 104 transmits instructions to the insulin pump 202 or 204, which delivers insulin to the patient 100. Insulin can be delivered in the form of a bolus dose, which raises the amount of insulin in the blood of the patient 100 by a predetermined amount. Additionally, insulin can be delivered in a scheduled manner in the form of a basal dose, which maintains a predetermined insulin level in the blood of the patient 100.

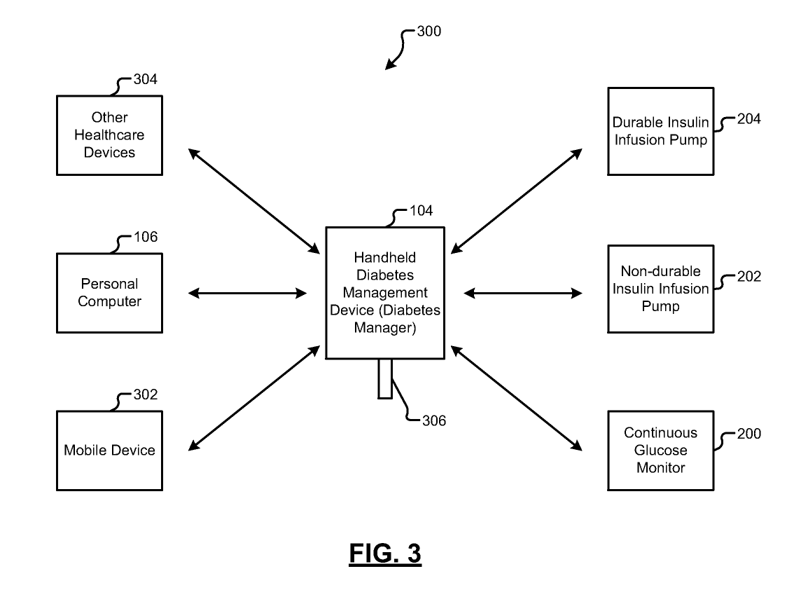

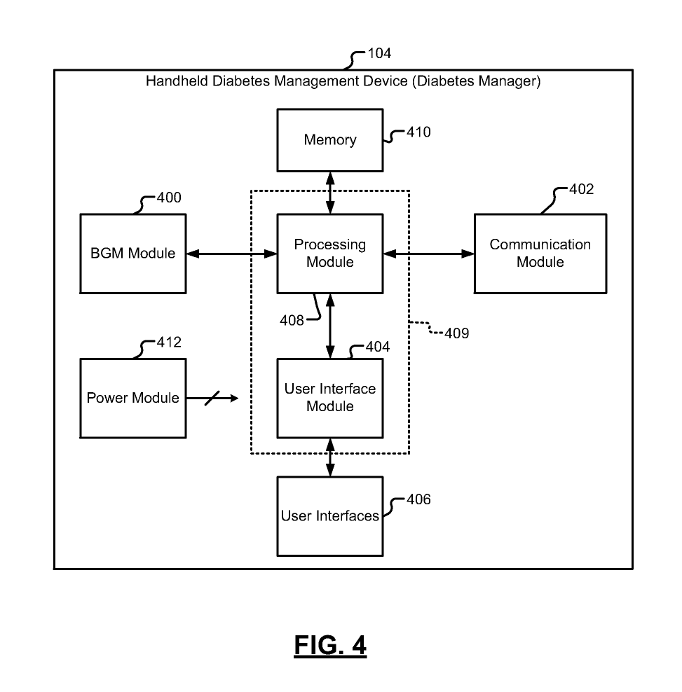

Referring now to FIG. 3, a diabetes management system 300 used by the patient 100 and the clinician 102 includes one or more of the following devices: the diabetes manager 104, the continuous glucose monitor (CGM) 200, the insulin pump 202 or 204, a mobile device 302, the diabetes analysis software on the PC 106, and other healthcare devices 304. The diabetes manager 104 is configured as a system hub and communicates with the devices of the diabetes management system 300. Alternatively, the insulin pump 204 or the mobile device 302 can serve as the system hub. Communication between the various devices in the diabetes management system 300 can be performed using wireless interfaces (e.g., Bluetooth) and/or wireline interfaces (e.g., USB). Communication protocols used by these devices can include protocols compliant with the IEEE 11073 standard as extended using guidelines provided by Continua® Health Alliance Design Guidelines. Further, healthcare records systems such as Microsoft® HealthVault™ and Google™ Health can be used by the patient 100 and clinician 102 to exchange information.

The diabetes manager 104 can receive glucose readings from one or more sources (e.g., from the CGM 200). The CGM 200 continuously measures the glucose level of the patient 100. The CGM 200 periodically communicates the glucose level to the diabetes manager 104. The diabetes manager 104 and the CGM 200 communicate wirelessly using a proprietary Gazell wireless protocol developed by Nordic Semiconductor, Inc.

Additionally, the diabetes manager 104 includes a blood glucose meter (BGM) and a port that communicates with the BGM (both not shown). The port can receive a blood glucose measurement strip 306. The patient 100 deposits a sample of blood or other bodily fluid on the blood glucose measurement strip 306. The BGM analyzes the sample and measures the blood glucose level in the sample. The blood glucose level measured from the sample and/or the glucose level read by the CGM 200 can be used to determine the amount of insulin to be administered to the patient 100.

The diabetes manager 104 communicates with the insulin pump 202 or 204. The insulin pump 202 or 204 can be configured to receive instructions from the diabetes manager 104 to deliver a predetermined amount of insulin to the patient 100. Additionally, the insulin pump 202 or 204 can receive other information including meal and/or exercise schedules of the patient 100. The insulin pump 202 or 204 can determine the amount of insulin to administer based on the additional information.

The insulin pump 202 or 204 can also communicate data to the diabetes manager 104. The data can include amounts of insulin delivered to the patient 100, corresponding times of delivery, and pump status. The diabetes manager 104 and the insulin pump 202 or 204 can communicate using a wireless communication protocol such as Bluetooth. Other wireless or wireline communication protocols can also be used.

In addition, the diabetes manager 104 can communicate with other healthcare devices 304. For example, the other healthcare devices 304 can include a blood pressure meter, a weight scale, a pedometer, a fingertip pulse oximeter, a thermometer, etc. The other healthcare devices 304 obtain and communicate personal health information of the patient 100 to the diabetes manager 104 through wireless, USB, or other interfaces. The other healthcare devices 304 use communication protocols compliant with ISO/IEEE 11073 extended using guidelines from Continual® Health Alliance. The diabetes manager 104 can communicate with the other healthcare devices 304 using interfaces including Bluetooth, USB, etc. Further, the devices of the diabetes management system 300 can communicate with each other via the diabetes manager 104.

The diabetes manager 104 can communicate with the PC 106 using Bluetooth, USB, or other interfaces. A diabetes management software running on the PC 106 includes an analyzer-configurator that stores configuration information of the devices of the diabetes management system 300. The configurator has a database to store configuration information of the diabetes manager 104 and the other devices. The configurator can communicate with users through standard web or computer screens in non-web applications. The configurator transmits user-approved configurations to the devices of the diabetes management system 300. The analyzer retrieves data from the diabetes manager 104, stores the data in a database, and outputs analysis results through standard web pages or computer screens in non-web based applications.

The diabetes manager 104 can communicate with the mobile device 302 using Bluetooth. The mobile device 302 can include a cellular phone, a PDA, or a pager. The diabetes manager 104 can send messages to an external network through the mobile device 302. The mobile device 302 can transmit messages to the external network based on requests received from the diabetes manager 104.

The CGM 200 uses a subcutaneous sensor to sense and monitor a physical attribute related to the glucose level of the patient 100. In some embodiments, the CGM 200 measures the level of glucose in the interstitial fluid of the patient 100, which is related to the glucose level of the patient 100. The level of glucose in the interstitial fluid of the patient 100 may be sensed by the CGM 200 by sampling an electrical characteristic, such as current. The sampled current, and therefore the level of glucose in the interstitial fluid, is related to the glucose level of the patient 100. In order to accurately estimate the glucose level of the patient 100 based on the physical attribute (current, etc.) measured by the CGM 200, the diabetes manager 104 can be periodically calibrated. While the remainder of this description is related to associating a current sensed by the CGM 200 to an estimated glucose level of the patient 100, one skilled in the art will appreciate that any physical attribute related to the glucose level of the patient 100 may be utilized instead.

The diabetes manager 104 can be calibrated by determining calibration data based on at least one current sample and at least one blood glucose measurement. The calibration data can take many forms, but is essentially data sufficient to convert the current sampled by the CGM 200 to an estimated glucose level of the patient 100. The current sampled by the CGM 200 and the glucose level of the patient 100 can be assumed to have a linear relationship within a normal measurement region of approximately 40 to 400 Milligrams per Deciliter. Based on this assumed linear relationship, the calibration data can be data sufficient to identify a linear equation that associates one or more current samples with an estimated glucose level of the patient. For example, the calibration data can be one or more coefficients of a linear equation. After calibration, the diabetes manager 104 can determine an estimated glucose level of the patient 100 based on the calibration data and the current sampled by the CGM 200.

Referring now to FIG. 4, an exemplary diabetes manager 104 includes a blood glucose measuring (BGM) module 400, a communication module 402, a user interface module 404, user interfaces 406, a processing module 408, memory 410, and a power module 412. The user interface module 404 and the processing module 408 can be implemented by an application processing module 409. The BGM module 400 includes a blood glucose measuring engine that analyzes samples provided by the patient 100 on the blood glucose measurement strip 306 and that measures the amount of blood glucose in the samples. The communication module 402 can include multiple radios that communicate with different devices of the diabetes management system 300. The user interface module 404 connects the diabetes manager 104 to various user interfaces 406 that the patient 100 can use to interact with the diabetes manager 104. For example, the user interfaces 406 can include keys, switches, a display, a speaker, a microphone, a secure digital (SD) card port, and/or a USB port (all not shown).

The processing module 408 processes data received from the BGM module 400, the communication module 402, and the user interface module 404. The processing module 408 uses memory 410 for processing and storing data. The memory 410 can include volatile and nonvolatile memory. The processing module 408 outputs data to and receives data from the user interfaces 406 via the user interface module 404. The processing module 408 outputs data to and receives data from the devices of the diabetes management system 300 via the communication module 402. The power module 412 supplies power to the components of the diabetes manager 104. The power module 412 can include a rechargeable battery or other source of power. The battery can be recharged, e.g., by using an adapter that plugs into a wall outlet and/or via a USB port on the diabetes manager 104.

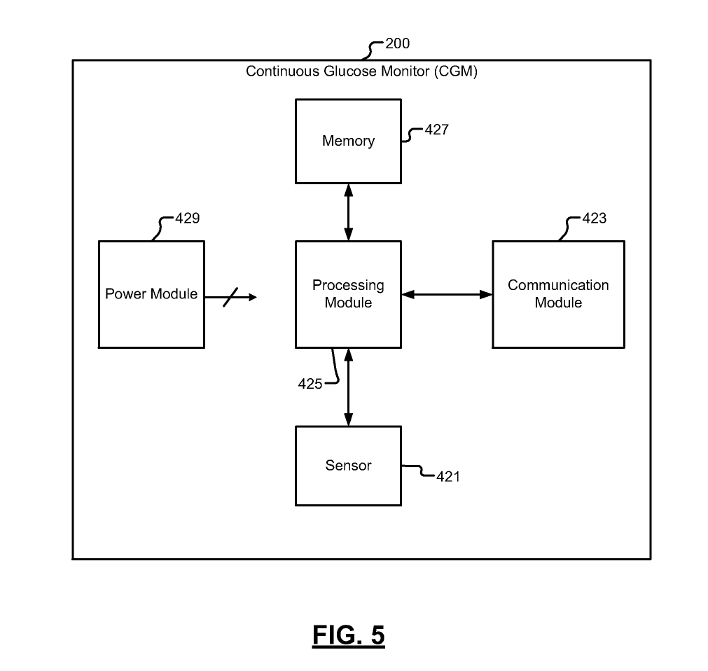

Referring now to FIG. 5, an exemplary continuous glucose monitor (CGM) 200 includes a sensor 421, a communication module 423, a processing module 425, memory 427, and a power module 429. The sensor 421 can monitor a condition of the patient 100 that is related to the glucose level of the patient 100. For example, the sensor 421, alone or in combination with processing module 425, can periodically sample a current value that corresponds to the level of glucose in the interstitial fluid of the patient 100. The communication module 423 can include one or more radios that communicate with different devices of the diabetes management system 300.

The processing module 425 processes data received from the sensor 421 and the communication module 423. The processing module 425 uses memory 427 for processing and storing data. The memory 427 can include volatile and nonvolatile memory. The memory 427 can be utilized to store information related to the configuration of the CGM 200, for example, definitions of measuring duration, failsafe limits and mathematical definitions and settings. The processing module 425 outputs data to and receives data from the devices of the diabetes management system 300 via the communication module 423. The power module 429 supplies power to the components of the CGM 200. In some embodiments, the power module 429 includes a battery or other source of power. The source of power may include a battery that can be recharged, e.g., by using an adapter that plugs into a wall outlet.

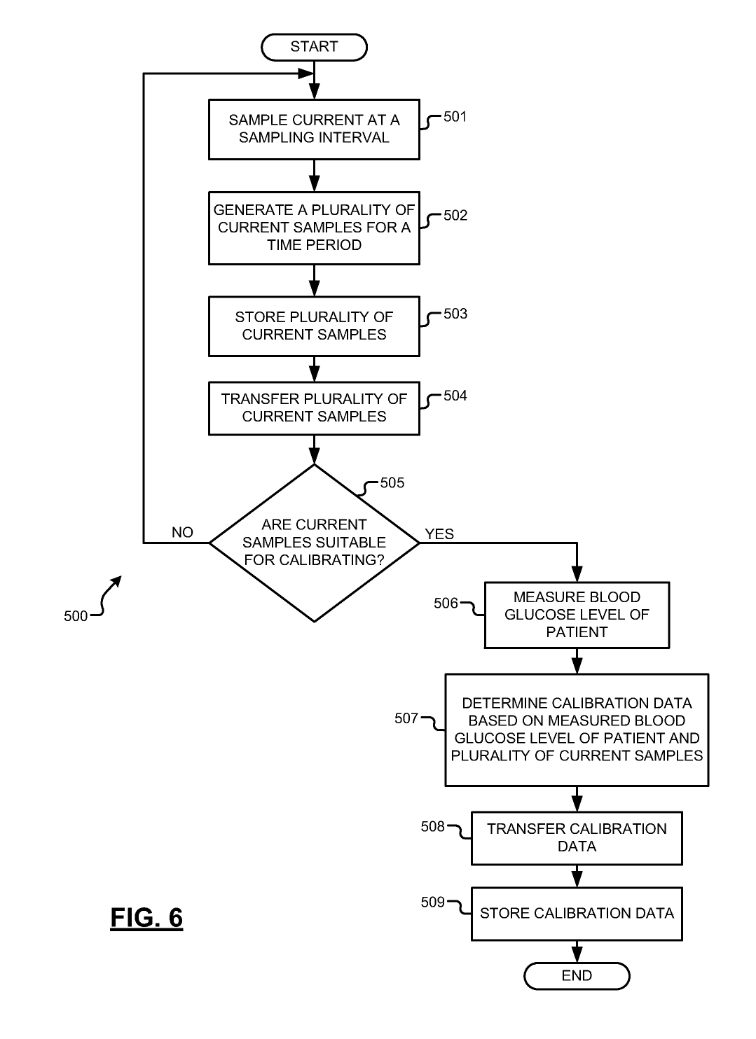

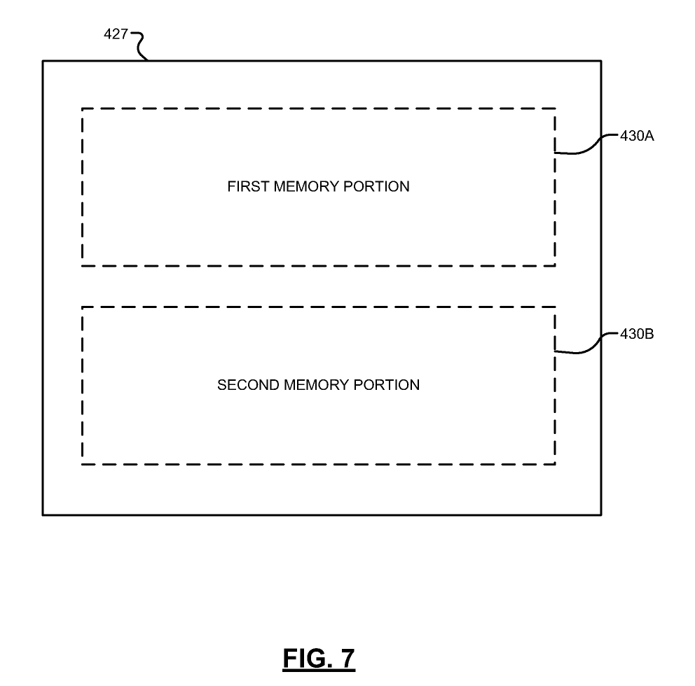

Referring now to FIG. 6, an exemplary method 500 of storing data at a continuous glucose monitor (CGM) 200 according to the present disclosure illustrated. The method 500 can permit a separate device to determine an estimated glucose level of a patient 100 based on the data stored at the CGM 200. The method 500 begins at step 501 where CGM 200 samples a current related to the glucose level of the patient 100 at a sampling interval. As described above, the current can be a measurement of the glucose level of the interstitial fluid of the patient 100, which in turn is related to the glucose level of the patient. For example only, the sampling interval can be one second, i.e., the CGM 200 can measure the current once per second. At step 502, the CGM 200 can generate a plurality of current samples for a time period. In one example, if the sampling interval is one second and the time period is one minute, the CGM 200 will generate sixty current samples per time period. At step 503 the plurality of current samples are stored at the CGM 200, for example, in memory 427, and at step 504 the plurality of current samples are transmitted to the diabetes manager 104 by the CGM 200.

In order to reduce the amount of information stored by the CGM 200 and/or transferred to the diabetes manager 104, the plurality of current samples can be preprocessed. The CGM 200 may preprocess the plurality of current samples for the time period by determining one or more statistical values from the plurality of current samples. The statistical values can be representative of the plurality of current samples. Examples of statistical values include, but are not limited to, the mean, the median, the standard deviation, the 25% quantile and the 75% quantile of the plurality of current samples. Further statistical values can also be utilized by the calibration method, such as a trend measure that corresponds to the change in the current samples over the time period. The trend measure can be utilized to indicate a direction and rate of change in the plurality of current samples. In this manner, the CGM 200 and/or diabetes manager 104 can store the statistical value(s) that are representative of the plurality of current samples for a time period, which can reduce the amount of data to be stored and transmitted. Furthermore, the statistical value(s) can be utilized by the CGM 200 and/or diabetes manager 104 for calibration purposes.

The plurality of current samples may contain erroneous or faulty measurements. For example, the current measured by the CGM 200 may contain sensor “noise” that causes a measured current sample to deviate from the actual glucose level of the patient 100. Such “noise” can be caused by, inter alia, physical movement of the CGM 200 relative to the patient 100 and/or electrical noise inherent within the CGM 200. Further, the CGM 200 may malfunction from time to time such that one or more current samples is substantially different from the actual glucose level of a patient 100, e.g., due to an internal issue in the electronics of the CGM 200 or sensor “dropout.” Sensor “dropout” can occur due to physiological problems with the attachment of the CGM 200 to the patient 100, e.g., physical movement of the CGM 200 relative to the patient 100, such that one or more current samples “drop” to near zero even when the actual glucose level of the patient 100 is higher.

The method proceeds to step 505 at which the diabetes manager 104, alone or in combination with the CGM 200, determines whether the plurality of current samples is suitable for calibrating the diabetes manager 104. In some embodiments, the suitability for calibration of a plurality of current samples can be determined by the absence of sensor “noise” and/or “dropout” from the current samples. Sensor “noise” and/or “dropout” can be detected in many ways. For example only, a high rate of variability in the current samples over a time period can be indicative of sensor “noise” and/or “dropout.” Therefore, different methods of determining a high rate of variability in the current samples can be utilized to determine the suitability of the current samples for calibration.

One method of determining whether the plurality of current samples is suitable for calibration is to compare the absolute value of the difference between the mean and median of the plurality of current samples with a threshold. In the event that the absolute value of the difference between the mean and median of the plurality of current samples is less than the threshold, the plurality of current samples can be deemed suitable for calibration. Similarly, in the event that the absolute value of the difference between the mean and median of the plurality of current samples is greater than the threshold, the plurality of current samples can be deemed unsuitable for calibration. This threshold can be set, for example, based on empirical data.

Another method of determining whether the plurality of current samples is suitable for calibration is to compare the standard deviation of the plurality of current samples with a threshold. In the event that the standard deviation of the plurality of current samples is less than the threshold, the plurality of current samples can be deemed suitable for calibration. Similarly, in the event that the standard deviation of the plurality of current samples is greater than the threshold, the plurality of current samples can be deemed unsuitable for calibration. This threshold can be set, for example, based on empirical data.

Yet another method of determining whether the plurality of current samples is suitable for calibration is to compare the median minus the 25% quantile value of the plurality of current samples with a threshold. In the event that the median minus the 25% quantile value of the plurality of current samples is less than the threshold, the plurality of current samples can be deemed suitable for calibration. Similarly, in the event that the median minus the 25% quantile value of the plurality of current samples is greater than the threshold, the plurality of current samples can be deemed unsuitable for calibration. This threshold can be set, for example, based on empirical data.

A further method of determining whether the plurality of current samples is suitable for calibration is to compare the 75% quantile value minus the median of the plurality of current samples with a threshold. In the event that the 75% quantile value minus the median of the plurality of current samples is less than the threshold, the plurality of current samples can be deemed suitable for calibration. Similarly, in the event that the 75% quantile value minus the median of the plurality of current samples is greater than the threshold, the plurality of current samples can be deemed unsuitable for calibration. This threshold can be set, for example, based on empirical data.

An additional method of determining whether the plurality of current samples is suitable for calibration is to compare the absolute value of a trend measure of the plurality of current samples with a threshold. The trend measure can correspond to the change in the current samples over the time period and can be a measure of a direction and rate of change in the plurality of current samples. A large trend measure may be indicative of a high rate of variability in a plurality of current samples. The trend measure can be determined by the following equation:

$${a}_{Trend} := \frac{{\sum }_{i = 1}^{N} ( {t}_{i} - \overline{t} ) ( {y}_{i} - \overline{y} )}{{\sum }_{i = 1}^{N} {( {t}_{i} - \overline{t} )}^{2}}$$

wherein i=1, 2, . . . n where n is a number of samples in the time period; yᵢ is the current at time i; y̅ is the mean over the time period; tᵢ is time at time i and the t̅ is a mean of the time period. In the event that the absolute value of the trend measure of the plurality of current samples is less than the threshold, the plurality of current samples can be deemed suitable for calibration. Similarly, in the event that the absolute value of the trend measure of the plurality of current samples is greater than the threshold, the plurality of current samples can be deemed unsuitable for calibration. This threshold can be set, for example, based on empirical data.

While each of the methods discussed above has been described as independently determining whether a plurality of current samples is suitable for calibration, it should be appreciated that these methods can also be utilized in combination with each other. For example only, the suitability of a plurality of current samples for calibration can be determined by comparing the standard deviation of the plurality of current samples with a first threshold and by comparing the absolute value of the difference between the mean and median of the plurality of current samples with a second threshold. In the event that the standard deviation of the plurality of current samples is less than the first threshold and the absolute value of the difference between the mean and median of the plurality of current samples is less than a second threshold, the plurality of current samples can be deemed suitable for calibration. Similarly, in the event that the standard deviation of the plurality of current samples is greater than the threshold or the absolute value of the difference between the mean and median of the plurality of current samples is greater than the second threshold, the plurality of current samples can be deemed unsuitable for calibration.

If the plurality of current samples is not deemed suitable for calibration at step 505, the method 500 does not determine calibration data based on the plurality of current samples and returns to step 501. If, however, the plurality of current samples is determined to be suitable for calibration at step 505, the method 500 proceeds to step 506 at which the blood glucose level of the patient 100 is measured, e.g., by the diabetes manager 104. The diabetes manager 104 can provide an indication to the patient 100 that a blood glucose measurement is desired for calibration, e.g., by a visual, tactile and/or audible alarm. Typically, the patient 100 would then measure his or her blood glucose level by depositing a sample of blood or other bodily fluid on the blood glucose measurement strip 306 to be analyzed by the BGM module 400 associated with the diabetes manager 104, although other methods of blood glucose level measurement could be utilized.

After measuring the blood glucose level of the patient 100 at step 506, the diabetes manager 104 can determine calibration data based on the measured blood glucose level of the patient 100 and the plurality of current samples (step 507). In order to increase the accuracy of the calibration data, the time at which the measurement of the blood glucose level of the patient 100 is taken (time of measurement) can correspond to the time period during which the plurality of current samples was sampled. It should be noted, however, that the time of measurement may not fall within the time period due to delay in the physiologic response of the patient 100, unsuitability of current samples for a time period, etc.

The calibration data can be determined in a variety of ways. For example, if one assumes a linear relationship between the current sampled by the CGM 200 and the blood glucose level of the patient 100, the calibration data can be one or more coefficients of a linear equation that are determined by applying a linear regression algorithm to the various data samples, i.e., the collection of measured blood glucose level/measured current pairs. The diabetes manager 104 can determine the calibration data based on one measured blood glucose level/measured current associates pair by utilizing a predetermined reference pair (such as [0,0] for measured blood glucose level/measured current). Furthermore, as the diabetes manager 104/CGM 200 accumulates a number of calibration reference points (that is, measured blood glucose level/measured current pairs) these additional reference points can be utilized, in conjunction with or instead of the predetermined reference pair, to more accurately calibrate the diabetes manager 104. One skilled in the art will appreciate, however, that alternative techniques can be used by the diabetes manager 104 to determine the calibration data. The calibration data is configured to allow the diabetes manager 104 to determine the estimated glucose level of the patient 100 based on the plurality of current samples. The calibration data can be stored in memory 410, and may be utilized by application processing module 409 to determine the estimated glucose level of the patient 100.

In the event that the diabetes manager 104 malfunctions or the calibration data stored in memory 410 otherwise becomes unavailable (through data corruption, loss of the diabetes manager 104, etc.), the plurality of current samples stored by the CGM 200 may be insufficient, by themselves, to determine the estimated glucose level of the patient 100. Furthermore, it may be advantageous to allow a separate device (not shown), such as a personal computer (at a patient's home, a doctor's office, etc.) or an additional diabetes managing device (similar to diabetes manager 104) to determine the estimated glucose level of the patient 100 from the plurality of current samples without requiring a transmission of the calibration data from the diabetes manager 104. Accordingly, in addition to storing the calibration data at the diabetes manager 104, the calibration data may be separately stored by the CGM 200, as described more fully below.

At step 508, the diabetes manager 104 transmits the calibration data to the CGM 200. The CGM 200 then stores the calibration data at step 509. The transmission of the calibration data to the CGM 200 (step 508) may be performed any time after the diabetes manager 104 determines the calibration data. For example, the diabetes manager 104 may transmit the calibration data to the CGM 200 immediately after it is determined. In this manner, the calibration data present at CGM 200 may always match the calibration data at the diabetes manager 104 (excluding the delay or an error associated with the transmission). If desired, the calibration data and the plurality of current samples stored at CGM 200 can be transmitted to the separate device such that the separate device can determine the estimated glucose level of the patient 100 based on the calibration data and the plurality of current samples received from the CGM 200.

In some embodiments, the CGM 200 will store a plurality of calibration data sets, each of which corresponding to a determination of calibration data by the diabetes manager 104 at step 507. In this manner, the CGM 200 can maintain a historical record of calibration data and current samples such that previously performed glucose level estimations can be reproduced. Accordingly, the CGM 200 may associate calibration data (or a calibration data set) with the plurality of current samples upon which such calibration data is based. In one example, the CGM 200 associates calibration data (or a calibration data set) with the plurality of current samples based on when the calibration data was determined at step 507. This may be performed, for example, by associating each of the plurality of current samples with a time indicator when the current was sampled at step 501. The time indicator can also be associated with the calibration data based on when the calibration data was determined, e.g., when step 507 is performed. In this manner, a current sample (or plurality of current samples) can be associated with calibration data (a calibration data set) by comparing their respective time indicators.

In order to permit the separate device to properly receive and distinguish between the plurality of current samples and calibration data, the different data types may be segregated and stored in different memory portions of the CGM 200. Referring now to FIG. 7, a functional block diagram of memory 427 of CGM 200 is illustrated. Memory 427 can include a first memory portion 430A and a second memory portion 430B. In one example, the plurality of current samples is stored in the first memory portion 430A and the calibration data is stored in the second memory portion 430B. The separate device can request the data in the first memory portion 430A if it desires to receive a current sample(s) or the second memory portion 430B if it desires the calibration data. One skilled in the art will appreciate that, instead of segregating the two data types, the CGM 200 can indicate the data type, i.e., whether the data is current sample(s) or calibration data, by marking the data with a label or marker that indicates the data type, utilizing a pointer, etc. In this manner, the separate device may send a request for either a current sample(s) or calibration data to the CGM 200 and the CGM 200 can retrieve and transmit the appropriate data set to the separate device.

The CGM 200 can interface and share information with the separate device using wireless interfaces (e.g., Bluetooth) and/or wireline interfaces (e.g., USB). Communication protocols used by these devices can include protocols compliant with the IEEE 11073 standard as extended using guidelines provided by Continua® Health Alliance Design Guidelines. In one example in which the separate device is a second handheld diabetes managing device, the separate device and the CGM 200 can communicate wirelessly using a proprietary Gazell wireless protocol developed by Nordic Semiconductor, Inc. A communication path can be established between the separate device and the CGM 200, for example by a wireline interface or establishing a wireless connection. Once a communication path has been established between the CGM 200 and the separate device the CGM 200 can transmit the plurality of current samples and/or the calibration data to the separate device by utilizing this communication path.

The broad teachings of the disclosure can be implemented in a variety of forms. Therefore, while this disclosure includes particular examples, the true scope of the disclosure should not be so limited since other modifications will become apparent to the skilled practitioner upon a study of the drawings, the specification, and the following claims.

This detailed description is merely illustrative in nature and is in no way intended to limit the disclosure, its application, or uses. For purposes of clarity, the same reference numbers are used in the drawings to identify similar elements. As used herein, the phrase at least one of A, B, and C should be construed to mean a logical (A or B or C), using a non-exclusive logical or. It should be understood that steps within a method can be executed in different order without altering the principles of the present disclosure.

As used herein, the term module can refer to, be part of, or include an Application Specific Integrated Circuit (ASIC); an electronic circuit; a combinational logic circuit; a field programmable gate array (FPGA); a processor (shared, dedicated, or group) that executes code; other suitable components that provide the described functionality; or a combination of some or all of the above, such as in a system-on-chip. The term module can include memory (shared, dedicated, or group) that stores code executed by the processor.

The term code, as used above, can include software, firmware, and/or microcode, and can refer to programs, routines, functions, classes, and/or objects. The term shared, as used above, means that some or all code from multiple modules can be executed using a single (shared) processor. In addition, some or all code from multiple modules can be stored by a single (shared) memory. The term group, as used above, means that some or all code from a single module can be executed using a group of processors. In addition, some or all code from a single module can be stored using a group of memories.

The apparatuses and methods described herein can be implemented by one or more computer programs or applications executed by one or more processors. The computer programs and applications can include processor-executable instructions that are stored on a non-transitory tangible computer readable medium. The computer programs can also include stored data. Non-limiting examples of the non-transitory tangible computer readable medium are nonvolatile memory, magnetic storage, and optical storage.

1. A method for storing data at a continuous glucose monitor and a handheld diabetes managing device such that an estimated glucose level of a patient can be determined by a third device from the data stored at the continuous glucose monitor, comprising:
measuring a blood glucose level of the patient;
receiving a plurality of physical attribute samples related to the glucose level of the patient;
determining calibration data based on the measured blood glucose level and at least one of the plurality of physical attribute samples, the calibration data configured to allow the handheld diabetes managing device to determine the estimated glucose level of the patient based on the plurality of physical attribute samples; and
transmitting the calibration data from the handheld diabetes managing device to the continuous glucose monitor for storage at the continuous glucose monitor.

2. The method of claim 1, further comprising:
sampling the plurality of physical attribute samples with the continuous glucose monitor;
storing the plurality of physical attribute samples at the continuous glucose monitor; and
storing the calibration data at the continuous glucose monitor.

3. The method of claim 2, wherein storing the plurality of physical attribute samples at the continuous glucose monitor includes associating each of the plurality of physical attribute samples with a time indicator.

4. The method of claim 3, wherein storing the calibration data at the continuous glucose monitor includes associating the calibration data with one of the time indicators based on when the calibration data was determined.

5. The method of claim 1, further comprising associating the calibration data with the plurality of physical attribute samples based on when the calibration data was determined.

6. The method of claim 1, wherein the plurality of physical attribute samples comprise a plurality of current samples sensed by the continuous glucose monitor.

7. The method of claim 1, wherein the calibration data includes coefficients of a linear equation.

8. The method of claim 1, further comprising storing the calibration data at the handheld diabetes managing device.

9. A method for storing data at a continuous glucose monitor and a handheld diabetes managing device such that an estimated glucose level of a patient can be determined by a third device from the data stored at the continuous glucose monitor, comprising:
sampling a physical attribute related to a glucose level of the patient with the continuous glucose monitor to generate a plurality of physical attribute samples;
storing the plurality of physical attribute samples at the continuous glucose monitor;
associating each of the plurality of physical attribute samples with a time indicator;
measuring a glucose level of the patient with the handheld diabetes managing device;
transmitting the plurality of physical attribute samples to the handheld diabetes managing device;
determining calibration data at the handheld diabetes managing device based on the measured blood glucose level and at least one of the plurality of physical attribute samples, the calibration data configured to allow the handheld diabetes managing device to determine the estimated glucose level of the patient based on the plurality of physical attribute samples;
transmitting the calibration data from the handheld diabetes managing device to the continuous glucose monitor;
storing the calibration data at the continuous glucose monitor;
transmitting the calibration data to the third device from the continuous glucose monitor;
transmitting the plurality of physical attribute samples to the third device from the continuous glucose monitor; and
determining the estimated glucose level of the patient at the third device based on the plurality of physical attribute samples and the calibration data.

10. The method of claim 9, further comprising associating the calibration data with the plurality of physical attribute samples based on when the calibration data was determined.

11. The method of claim 9, wherein the plurality of physical attribute samples comprise a plurality of current samples sensed by the continuous glucose monitor.

12. The method of claim 9, wherein the calibration data includes coefficients of a linear equation.

13. The method of claim 9, further comprising storing the calibration data at the handheld diabetes managing device.

14. The method of claim 9, wherein storing the calibration data at the continuous glucose monitor includes associating the calibration data with one of the time indicators based on when the calibration data was determined.

15. A diabetes management system that allows a separate device to determine an estimated glucose level of a patient, comprising:
a continuous glucose monitor including a memory and configured to: (i) sample a physical attribute related to a glucose level of the patient to generate a plurality of physical attribute samples, (ii) store the plurality of physical attribute samples and (iii) store calibration data configured to allow the separate device to determine the estimated glucose level of the patient based on the plurality of physical attribute samples; and
a handheld diabetes managing device in communication with the continuous glucose monitor and configured to: (i) receive the plurality of physical attribute samples from the continuous glucose monitor, (ii) measure a blood glucose level of the patient, (iii) determine the calibration data based on the measured blood glucose level and at least one of the plurality of physical attribute samples, and (iv) transmit the calibration data to the continuous glucose monitor for storage.

16. The diabetes management system of claim 15, further comprising the separate device, the separate device configured to: (i) receive the calibration data and the plurality of physical attribute samples from the continuous glucose monitor, and (ii) determine the estimated glucose level of the patient based on the plurality of physical attribute samples and the calibration data.

17. The diabetes management system of claim 15, wherein the handheld diabetes managing device is further configured to store the calibration data at the handheld diabetes managing device.

18. The diabetes management system of claim 15, wherein the continuous glucose monitor or the handheld diabetes managing device or the separate device is configured to associate the calibration data with the plurality of physical attribute samples based on when the calibration data was determined.

19. The diabetes management system of claim 15, wherein the plurality of physical attribute samples comprise a plurality of current samples sensed by the continuous glucose monitor.

20. The diabetes management system of claim 15, wherein the calibration data includes coefficients of a linear equation.
